(19) United States
(12) Patent Application Publication
(10) Pub. No.: US 2009/0005737 A1
(43) Pub. Date: Jan. 1, 2009

(54) Auto-Injector

(76) Inventor: Thomas Chun, Northridge, CA (US)

Correspondence Address: ITALIA IP, 3500 WEST OLIVE AVE., SUITE 300, BURBANK, CA, 91505, US

(21) Appl. No.: 11/770,738

(22) Filed: Jun. 29, 2007

(51) Int. Cl. A61M 5/20

(52) U.S. Cl. 604/157; 604/263

(57) ABSTRACT

An auto-injector that includes a hollow tubular shell to retain the internal structure and provide a surface to be held by the user; a trigger for activating the auto-injector; a plunger lock for slideably inserting into the trigger and for retaining a plunger in the operative position prior to use; and, a syringe assembly comprising a hollow tubular barrel for slideably receiving the plunger, such that when the trigger is activated the syringe assembly is activated and the injection is automatically administered.

### BACKGROUND

This application relates generally to an auto-injector. More specifically, this application relates to an auto-injector that allows a liquid drug to be introduced into the body through the skin.

### SUMMARY

This application discloses an auto-injector (also referred to herein as simply an injector) that acts through the push of a single button or trigger. The disclosed injector allows even an individual who is not accustomed to performing injections to do so. It is even possible to inject oneself with a liquid drug using the disclosed injector. The injector is economical to produce, of simple construction and capable of mass production.

In particular, this application discloses an auto-injector comprising: an upper and lower hollow tubular shell, wherein the upper tubular shell is open at one end and includes a cutout about its surface, and wherein the lower hollow tubular shell includes a lower opening at one end for the a needle to pass through and wherein the opposite end attaches to said upper shell to form a complete shell to retain the internal structure of the auto-injector and provide a surface to be held by the user; a trigger for slideably inserting into said upper shell, wherein the trigger includes a longitudinal bore therethrough and an upper retaining portion of a smaller diameter, a lower releasing portion of a larger diameter, and a button portion integral with said lower releasing portion and extending through said cutout of said upper shell for activating said auto-injector; a plunger lock for slideably inserting into said trigger, wherein the plunger lock includes a longitudinal bore therethrough, deflectable flange portions at one end for abutment to at least a portion of the upper retaining portion of said trigger prior to use, and a flared spring abutting portion; and, a syringe assembly comprising a hollow tubular barrel for slideably receiving a plunger, wherein the barrel further comprises a plunger receiving end and a needle attachment portion for attachment of a needle assembly at the other end, and wherein the plunger includes a firing rib about its surface to abut against a portion of the deflectable flange portions prior to use and a spring rib interior to said firing rib for attachment of a firing spring, wherein said firing spring includes a flared portion at one end for abutting to said spring abutting portion of said plunger lock.

In another embodiment, this application discloses an auto-injector comprising: an upper and lower hollow tubular shell, wherein the upper tubular shell is open at one end and includes a cutout about its surface, and wherein the lower hollow tubular shell includes a lower opening at one end for the a needle to pass through and wherein the opposite end attaches to said upper shell to form a complete shell to retain the internal structure of the auto injector and provide a surface to be held by the user; a trigger for slideably inserting into said upper shell, wherein the trigger includes a longitudinal bore therethrough and an upper retaining portion of a smaller diameter, a lower releasing portion of a larger diameter, and a button portion integral with said lower releasing portion and extending through said cutout of said upper shell for activating said auto-injector; a plunger lock for slideably inserting into said trigger, wherein the plunger lock includes a longitudinal bore therethrough, deflectable flange portions at one end for abutment to at least a portion of the upper retaining portion of said trigger prior to use, and a flared spring abutting portion; a syringe assembly comprising a hollow tubular barrel for slideably receiving a plunger, wherein the barrel further comprises a plunger receiving end and a needle attachment portion for attachment of a needle assembly at the other end, and wherein the plunger includes a firing rib about its surface to abut against a portion of the deflectable flange portions prior to use and a spring rib interior to said firing rib for attachment of a firing spring, wherein said firing spring includes a flared portion at one end for abutting to said spring abutting portion of said plunger lock; and, wherein the button portion of said trigger includes an angled edge such than when the button is pressed is a direction substantially perpendicular to the axis of the auto-injector the trigger moves upward within the upper shell in an direction substantially parallel to the axis of the auto-injector, and thereby causes the upper retaining portion of the trigger to move upward such that the deflectable flange portions are now positioned at the lower releasing portion and no longer abutting against the trigger, in this position the force in the extended firing spring overcomes the retaining force of the deflectable flanges, and as the firing spring contracts, the plunger attached thereto deflects the flanges and is pulled downward into the barrel; wherein the force created by the firing spring pulling the plunger down into the barrel further causes the syringe assembly to advance a sufficient distance within the lower shell such that a least a portion of the needle exits through the lower opening in said lower shell and the injection is administered.

In yet a further embodiment, this application discloses an auto-injector comprising: an upper and lower hollow tubular shell, wherein the upper tubular shell is open at one end and includes a cutout about its surface, and wherein the lower hollow tubular shell includes a lower opening at one end for the a needle to pass through and wherein the opposite end attaches to said upper shell to form a complete shell to retain the internal structure of the auto injector and provide a surface to be held by the user; a trigger for slideably inserting into said upper shell, wherein the trigger includes a longitudinal bore therethrough and an upper retaining portion of a smaller diameter, a lower releasing portion of a larger diameter, and a button portion integral with said lower releasing portion and extending through said cutout of said upper shell for activating said auto-injector; a plunger lock for slideably inserting into said trigger, wherein the plunger lock includes a longitudinal bore therethrough, deflectable flange portions at one end for abutment to at least a portion of the upper retaining portion of said trigger prior to use, and a flared spring abutting portion; a syringe assembly comprising a hollow tubular barrel for slideably receiving a plunger, wherein the barrel further comprises a plunger receiving end and a needle attachment portion for attachment of a needle assembly at the other end, and wherein the plunger includes a firing rib about its surface to abut against a portion of the deflectable flange portions prior to use and a spring rib interior to said firing rib for attachment of a firing spring, wherein said firing spring includes a flared portion at one end for abutting to said spring abutting portion of said plunger lock; wherein the button portion of said trigger includes an angled edge such than when the button is pressed is a direction substantially perpendicular to the axis of the auto-injector the trigger moves upward within the upper shell in an direction substantially parallel to the axis of the auto-injector, and thereby causes the upper retaining portion of the trigger to move upward such that the deflectable flange portions are now positioned at the lower releasing portion and no longer abutting against the trigger, in this position the force in the extended firing spring overcomes the retaining force of the deflectable flanges, and as the firing spring contracts, the plunger attached thereto deflects the flanges and is pulled downward into the barrel; wherein the force created by the firing spring pulling the plunger down into the barrel further causes the syringe assembly to advance a sufficient distance within the lower shell such that a least a portion of the needle exits through the lower opening in said lower shell and the injection is administered; a solute container attached at a position between the needle attachment portion and the needle assembly; and, a needle shield assembly that is activated after firing of the auto-injector.

### BRIEF DESCRIPTION OF THE DRAWINGS

The drawings, when considered in connection with the following description, are presented for the purpose of facilitating an understanding of the subject matter sought to be protected.

FIG. 1 is a front elevational view of a first embodiment of the auto-injector described herein shown prior to use;

FIG. 2 is a sectional view of the auto-injector taken generally along the line 2-2 in FIG. 1;

FIG. 3 is a front elevational view of the auto-injector shown in FIG. 1 rotated 90°;

FIG. 4 is a sectional view of the auto-injector taken generally along the line 4-4 in FIG. 3;

FIG. 5 is a front elevational view of the first embodiment of the auto-injector described herein shown after use;

FIG. 6 is a sectional view of the auto-injector taken generally along the line 6-6 in FIG. 5;

FIG. 7 is a front elevational view of the auto-injector shown in FIG. 5 rotated 90°;

FIG. 8 is a sectional view of the auto-injector taken generally along the line 8-8 in FIG. 7;

FIG. 9 is an exploded view of the auto-injector of FIG. 1;

FIG. 10 is enlarged fragmentary sectional view of the auto-injector in FIG. 2;

FIG. 11 is enlarged fragmentary sectional view of the auto-injector in FIG. 2;

FIG. 12 is enlarged fragmentary sectional view of the auto-injector in FIG. 2 shown with the needle cap attached;

FIG. 13 is a front elevational view of a second embodiment of the auto-injector described herein shown prior to use;

FIG. 14 is a sectional view of the auto-injector taken generally along the line 14-14 in FIG. 13;

FIG. 15 is a front elevational view of the auto-injector shown in FIG. 13 rotated 90°;

FIG. 16 is a sectional view of the auto-injector taken generally along the line 16-16 in FIG. 15;

FIG. 17 is a front elevational view of the second embodiment of the auto-injector described herein shown after use; and,

FIG. 18 is a sectional view of the auto-injector taken generally along the line 18-18 in FIG. 17;

### DETAILED DESCRIPTION

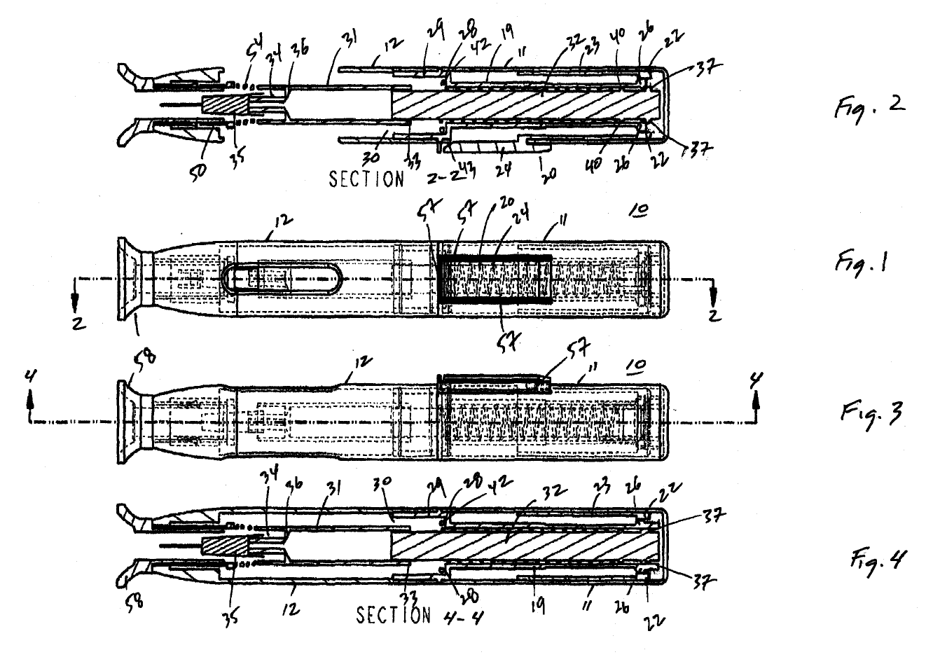

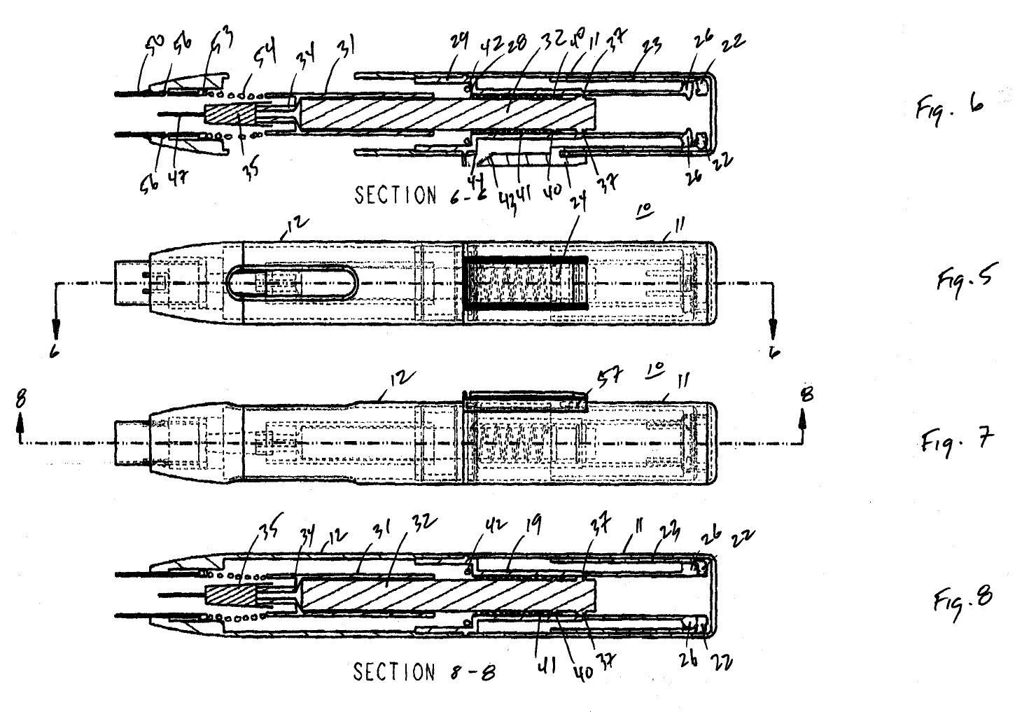

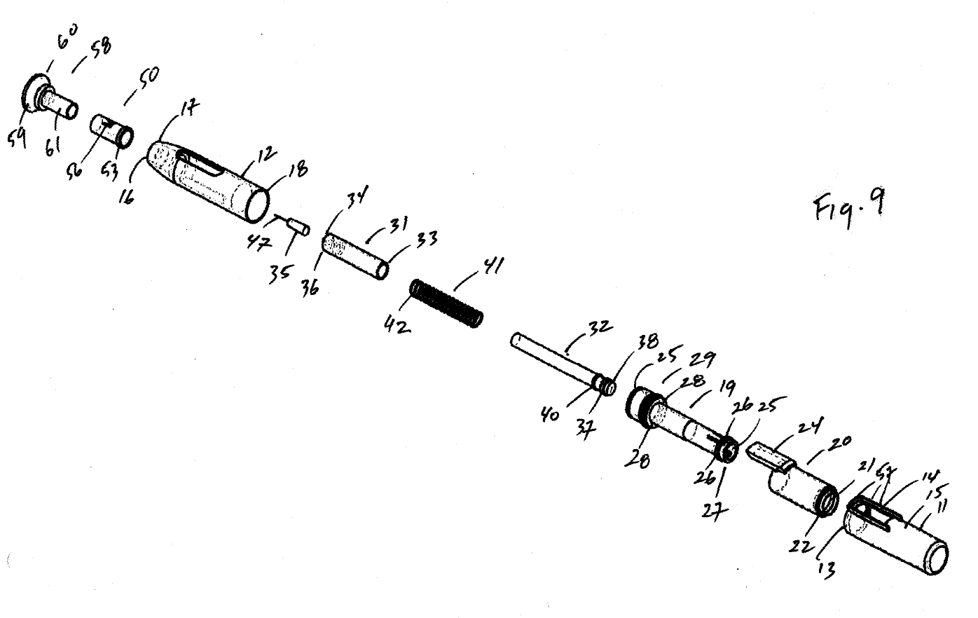

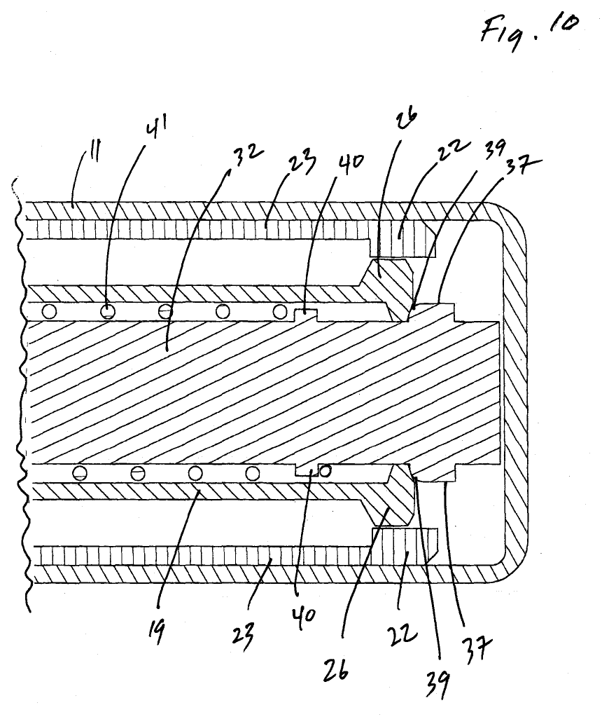

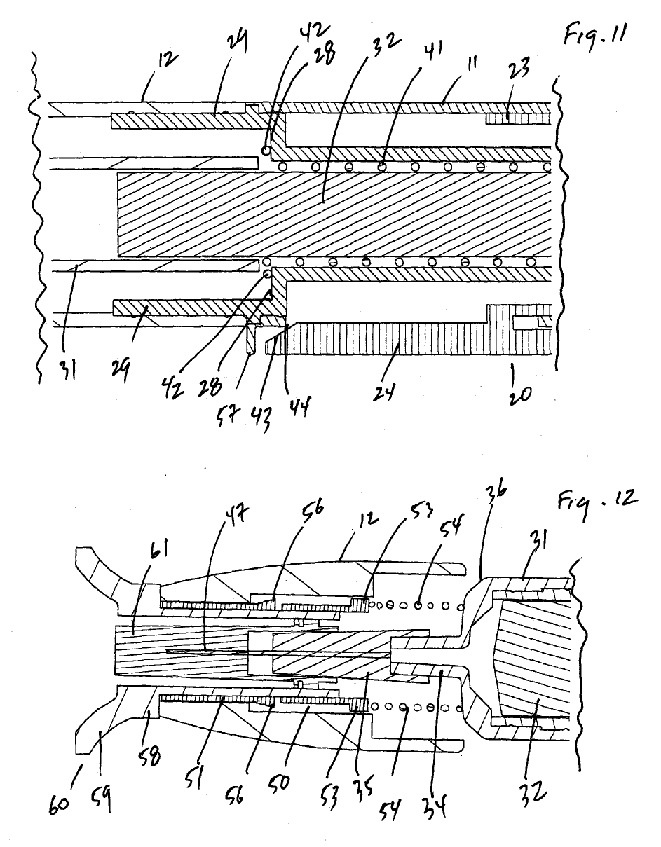

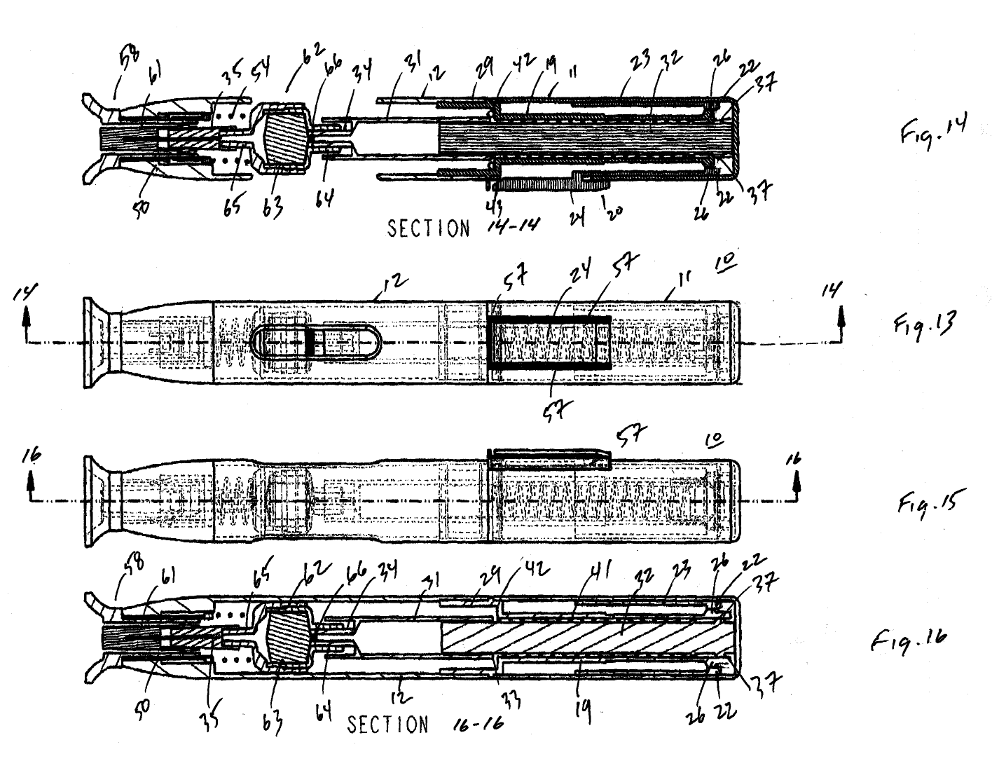

Referring to FIGS. 1-4 and 9, shown therein and generally designated by the reference character 10 is first embodiment of an auto-injector constructed in accordance with the following description shown prior to use or also referred to herein as in the “operative position.” The auto-injector 10 includes an upper 11 and lower 12 hollow tubular shell, wherein the upper tubular shell 11 is open at one end 13 and includes a cutout 14 about its surface 15, and wherein the lower hollow tubular shell 12 includes a lower opening 16 at one end 17 for the a needle 47 to pass through. The opposite end 18 attaches to the upper shell 11 to form a complete shell to retain the internal structure of the auto-injector and provide a surface to be held by the user.

The auto-injector also includes a trigger 20 for slideably inserting into the upper shell 11. The trigger 20 includes a longitudinal bore 21 therethrough and an upper retaining portion 22 of a smaller diameter, a lower releasing portion 23 of a larger diameter, and a button portion 24 integral with the lower releasing portion 23 and extending through the cutout 14 of the upper shell 11 for activating the auto-injector 10.

The auto-injector also includes a plunger lock 19 for slideably inserting into said trigger 20, wherein the plunger lock 19 includes a longitudinal bore 25 therethrough, a pair of deflectable flange portions 26 at one end 27 for abutment to at least a portion of the upper retaining portion 22 of said trigger 20 prior to use and thereby in the operative position, and a flared spring abutting portion 28 at the other end 29.

A syringe assembly 30 is included in the auto-injector and includes a hollow tubular barrel 31 for slideably receiving a plunger 32, wherein the barrel 31 further includes a plunger receiving end 33 and a needle attachment portion 34 for attachment of a needle assembly 35 at the other end 36. As a component of the firing mechanism of the auto-injector 10, the plunger 32 includes a firing rib 37 at one end 38 which when in the operative position prior to use abuts against a portion 39 of the deflectable flange portions 26. A spring rib 40 interior to the firing rib 37 is used for attachment of a firing spring 41. The firing spring 41 includes a flared portion 42 at one end for abutting to the spring abutting portion 28 of said plunger lock 19. The spring rib 40 is a single ribbed portion protruding around the outer circumference of the plunger 32. The firing spring 41 is retained upon the plunger 32 and more specifically, the spring rib 40, by butting one end of the firing spring 41 against the spring rib 40. This may be accomplished by screwing the one end of the firing spring 41 onto the surface of the plunger 32 such that the end of the firing spring 41 is retained on the opposite side of the spring rib 40 relative to the rest of the firing spring 41. See FIGS. 10 and 11. Because the firing spring 41 is held at both ends (the flared portion 42 at one end for abutting to the spring abutting portion 28 of said plunger lock 19 and upon the spring rib 40) the spring can be extended or loaded and the plunger locked into position as shown and described. The term “extended” or “loaded” refers to when a force created within the spring after an initial force is applied to the spring to lengthen it. More specifically, a recoiling force is created in the spring that will act to force the spring back to its non-lengthened state.

Referring now to FIGS. 5-8, shown therein and generally designated by the reference character 10 is the first embodiment of an auto-injector constructed in accordance with the following description shown after use. To “fire” the auto-injector 10, a user simply presses the button portion 24 of the trigger 20. Preferably the button includes an angled edge 43 of approximately 30° to 40° such than when the button 24 is pressed is a direction substantially perpendicular to the axis of the auto-injector 10 the angled edge engages a portion of 44 of the cutout 14 of the upper shell 11 thereby causing the trigger 20 to move upward within the upper shell 11 in an direction substantially parallel to the axis of the auto-injector 10. This advancement causes the upper retaining portion 22 of the trigger 20 to move upward such that the deflectable flange portions 26 of the plunger lock 19 are now positioned at the lower releasing portion 23 and no longer abutting against the trigger 20 and more specifically the upper retaining portion 22. In this position the force in the extended firing spring 41 overcomes the retaining force of the deflectable flanges 26 which are pressed out in a direction perpendicular to the auto-injector axis, thereby releasing the plunger 32, and as the firing spring 41 contracts, the plunger 32 attached thereto is pulled downward into the barrel 31. The force created by the firing spring 41 pulling the plunger 32 down into the barrel 31 further causes the syringe assembly 30 to advance a sufficient distance within the lower shell 12 such that a least a portion 46 of the needle 47 exits through the lower opening 16 in the lower shell 12 and the injection is administered. It should also be appreciated that any movement of the trigger 20, be it moving it in a downward direction, tilting it, sliding it, or an other movement, that acts to remove the retaining portion 22 from its abutting position with the deflectable flange portion 26 would also effect the firing of the injector 10.

Referring now to FIG. 12, the auto-injector 10 may also preferably include a needle shield assembly 50 that is activated after firing of the auto-injector 10. The needle shield assembly includes a shield portion 51 that is substantially circular with a longitudinal bore 52 therethrough and includes a flared portion 53 for abutting a second spring 54 at one end 55 and deflectable flange portions 56 about its surface. The second spring 54 is attached to the needle assembly 35 and is in a contracted position prior to use and after firing expands and thereby creates a force on the flared portion 53 of the shield 51 thereby pushing it outward until the deflectable flanges 56 expand and lock the shield 51 in the extended position thereby covering the portion 46 of the needle 47 exiting through the lower opening 16 in the lower shell 12.

The auto-injector may also include button protecting means for preventing the auto-injector from firing inadvertently such as when it is dropped, carried, shipped, and/or stored. The button protecting means includes at least one edge portion 57 (and preferably three) integral and extending from the surface of said cutout 14 of said upper shell 11. The edge portions 57 extend to a distance such that they are at least as high or higher than the button 24 integral with the trigger 20. Thus, for example, if the auto-injector is dropped and the button side contacts the ground, the edge portions 57 will absorb the impact and protect the button 24 from being engaged.

Further, the auto-injector may also include needle cap 58 that includes a flared portion 59 of increasing diameter toward one end 60 for engagement by the user during removal of the needle cap and a capping portion 61 that the frictionally fits upon the needle assembly 35.

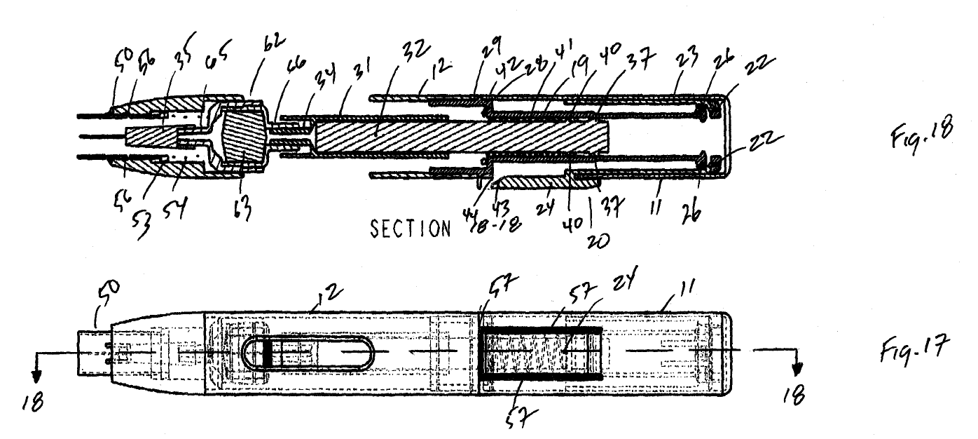

Finally, as shown in FIGS. 14-18, a second embodiment of the auto-injector 10 is shown wherein solvent container means is used. The functionality and operation is the same as described for the injector without the solvent container. Thus as shown, the auto-injector 10 may include a solute containing means such as a solute container 62 attached at a position between the needle attachment portion 34 and the needle assembly 35. The solute container 62 is utilized when the auto-injector is provided with a liquid solvent in the barrel 31 and a dry solute in the container 62 such that when the shot is administered, the solvent is combined with the solute such that an effective dose of the solute is administered by the user. The solute container 62 includes a substantially hollow main body 63 for holding the solute and a barrel attachment portion 64 at one end and a needle assembly attachment portion 65 at the other. The solute container 62 may also include structures such as channels of various shapes for providing that the solute is effectively solubilzed when the solvent is introduced. The auto-injector 10 may also include blocking means for preventing solvent retained within the barrel 31 from entering the solute container 50 prior to use. For example, the blocking means may be a ball 66 that is sized to be frictionally retained within said needle attachment portion 34 such that when sufficient force is applied by the plunger 32, the ball 66 is expelled from the needle attachment portion 34 and the solvent is able to enter the solvent container 62.

In the construction of the auto-injector 10, a majority of the components may be formed of suitable plastics that may be molded, however, components that must withstand load bearing and other forces such as the springs and needle may be formed of suitable metals.

While the present disclosure has been described in connection with what is considered the most practical and preferred embodiment, it is understood that this disclosure is not limited to the disclosed embodiments, but is intended to cover various arrangements included within the spirit and scope of the broadest interpretation so as to encompass all such modifications and equivalent arrangements.

What is claimed is:

1. An auto-injector comprising:
an upper and lower hollow tubular shell, wherein the upper tubular shell is open at one end and includes a cutout about its surface, and wherein the lower hollow tubular shell includes a lower opening at one end for the a needle to pass through and wherein the opposite end attaches to said upper shell to form a complete shell to retain the internal structure of the auto-injector and provide a surface to be held by the user;
a trigger for slideably inserting into said upper shell, wherein the trigger includes a longitudinal bore therethrough and an upper retaining portion of a smaller diameter, a lower releasing portion of a larger diameter, and a button portion integral with said lower releasing portion and extending through said cutout of said upper shell for activating said auto-injector;
a plunger lock for slideably inserting into said trigger, wherein the plunger lock includes a longitudinal bore therethrough, deflectable flange portions at one end for abutment to at least a portion of the upper retaining portion of said trigger prior to use, and a flared spring abutting portion; and,
a syringe assembly comprising a hollow tubular barrel for slideably receiving a plunger, wherein the barrel further comprises a plunger receiving end and a needle attachment portion for attachment of a needle assembly at the other end, and wherein the plunger includes a firing rib about its surface to abut against a portion of the deflectable flange portions prior to use and a spring rib interior to said firing rib for attachment of a firing spring, wherein said firing spring includes a flared portion at one end for abutting to said spring abutting portion of said plunger lock.

2. The auto-injector of claim 1 further comprising a solute container attached at a position between the needle attachment portion and the needle assembly.

3. The auto-injector of claim 2 further comprising a needle shield assembly that is activated after firing of the auto-injector.

4. The auto-injector of claim 3 further comprising button protecting means.

5. The auto-injector of claim 4 wherein the button protecting means includes at least one edge portion integral and extending from the surface of said cutout of said upper shell.

6. The auto-injector of claim 5 further comprising a needle cap that includes a flared portion of increasing diameter toward one end for engagement by the user during removal of the needle cap and a capping portion that frictionally fits upon said needle assembly.

7. The auto-injector of claim 1 wherein, in the operative position prior to use the firing spring is retained in an extended position around said plunger and between said spring rib on said plunger and said flared spring abutting portion of said plunger lock;

8. The auto-injector of claim 7 wherein the button portion of said trigger includes an angled edge such than when the button is pressed is a direction substantially perpendicular to the axis of the auto-injector the trigger moves upward within the upper shell in an direction substantially parallel to the axis of the auto-injector, and thereby causes the upper retaining portion of the trigger to move upward such that the deflectable flange portions are now positioned at the lower releasing portion and no longer abutting against the trigger, in this position the force in the extended firing spring overcomes the retaining force of the deflectable flanges, and as the firing spring contracts, the plunger attached thereto deflects the flanges and is pulled downward into the barrel.

9. The auto-injector of claim 8 wherein the force created by the firing spring pulling the plunger down into the barrel further causes the syringe assembly to advance a sufficient distance within the lower shell such that a least a portion of the needle exits through the lower opening in said lower shell and the injection is administered.

10. The auto-injector of claim 9 further comprising a needle shield assembly that is activated after firing of the auto-injector.

11. The auto-injector of claim 10 wherein the needle shield assembly includes a shield portion that is substantially circular with a longitudinal bore therethrough and includes a flared portion for abutting a second spring at one end and deflectable flange portions about its surface, wherein said second spring is attached to the needle assembly and is in a contracted position prior to use and after firing expands and thereby creates a force on the flared portion of the shield thereby pushing it outward until the deflectable flanges expand and lock the shield in the extended position thereby covering the portion of the needle exiting through the lower opening in said lower shell.

12. An auto-injector comprising:
an upper and lower hollow tubular shell, wherein the upper tubular shell is open at one end and includes a cutout about its surface, and wherein the lower hollow tubular shell includes a lower opening at one end for the a needle to pass through and wherein the opposite end attaches to said upper shell to form a complete shell to retain the internal structure of the auto injector and provide a surface to be held by the user;
a trigger for slideably inserting into said upper shell, wherein the trigger includes a longitudinal bore therethrough and an upper retaining portion of a smaller diameter, a lower releasing portion of a larger diameter, and a button portion integral with said lower releasing portion and extending through said cutout of said upper shell for activating said auto-injector;
a plunger lock for slideably inserting into said trigger, wherein the plunger lock includes a longitudinal bore therethrough, deflectable flange portions at one end for abutment to at least a portion of the upper retaining portion of said trigger prior to use, and a flared spring abutting portion;
a syringe assembly comprising a hollow tubular barrel for slideably receiving a plunger, wherein the barrel further comprises a plunger receiving end and a needle attachment portion for attachment of a needle assembly at the other end, and wherein the plunger includes a firing rib about its surface to abut against a portion of the deflectable flange portions prior to use and a spring rib interior to said firing rib for attachment of a firing spring, wherein said firing spring includes a flared portion at one end for abutting to said spring abutting portion of said plunger lock; and,
wherein the button portion of said trigger includes an angled edge such than when the button is pressed is a direction substantially perpendicular to the axis of the auto-injector the trigger moves upward within the upper shell in an direction substantially parallel to the axis of the auto-injector, and thereby causes the upper retaining portion of the trigger to move upward such that the deflectable flange portions are now positioned at the lower releasing portion and no longer abutting against the trigger, in this position the force in the extended firing spring overcomes the retaining force of the deflectable flanges, and as the firing spring contracts, the plunger attached thereto deflects the flanges and is pulled downward into the barrel; wherein the force created by the firing spring pulling the plunger down into the barrel further causes the syringe assembly to advance a sufficient distance within the lower shell such that a least a portion of the needle exits through the lower opening in said lower shell and the injection is administered.

13. The auto-injector of claim 12 further comprising a needle shield assembly that is activated after firing of the auto-injector.

14. The auto-injector of claim 12 further comprising a needle cap that includes a flared portion of increasing diameter toward one end for engagement by the user during removal of the needle cap and a capping portion the frictionally fits upon said needle assembly.

15. The auto-injector of claim 12 further comprising button protecting means.

16. The auto-injector of claim 15 wherein the button protecting means includes at least one edge portion integral and extending from the surface of said cutout of said upper shell.

17. An auto-injector comprising:
an upper and lower hollow tubular shell, wherein the upper tubular shell is open at one end and includes a cutout about its surface, and wherein the lower hollow tubular shell includes a lower opening at one end for the a needle to pass through and wherein the opposite end attaches to said upper shell to form a complete shell to retain the internal structure of the auto injector and provide a surface to be held by the user;
a trigger for slideably inserting into said upper shell, wherein the trigger includes a longitudinal bore therethrough and an upper retaining portion of a smaller diameter, a lower releasing portion of a larger diameter, and a button portion integral with said lower releasing portion and extending through said cutout of said upper shell for activating said auto-injector;
a plunger lock for slideably inserting into said trigger, wherein the plunger lock includes a longitudinal bore therethrough, deflectable flange portions at one end for abutment to at least a portion of the upper retaining portion of said trigger prior to use, and a flared spring abutting portion;
a syringe assembly comprising a hollow tubular barrel for slideably receiving a plunger, wherein the barrel further comprises a plunger receiving end and a needle attachment portion for attachment of a needle assembly at the other end, and wherein the plunger includes a firing rib about its surface to abut against a portion of the deflectable flange portions prior to use and a spring rib interior to said firing rib for attachment of a firing spring, wherein said firing spring includes a flared portion at one end for abutting to said spring abutting portion of said plunger lock;
wherein the button portion of said trigger includes an angled edge such than when the button is pressed is a direction substantially perpendicular to the axis of the auto-injector the trigger moves upward within the upper shell in an direction substantially parallel to the axis of the auto-injector, and thereby causes the upper retaining portion of the trigger to move upward such that the deflectable flange portions are now positioned at the lower releasing portion and no longer abutting against the trigger, in this position the force in the extended firing spring overcomes the retaining force of the deflectable flanges, and as the firing spring contracts, the plunger attached thereto deflects the flanges and is pulled downward into the barrel; wherein the force created by the firing spring pulling the plunger down into the barrel further causes the syringe assembly to advance a sufficient distance within the lower shell such that a least a portion of the needle exits through the lower opening in said lower shell and the injection is administered; and,
a solute container attached at a position between the needle attachment portion and the needle assembly.

18. The auto-injector of claim 17 further comprising a needle shield assembly that is activated after firing of the auto-injector.

19. The auto-injector of claim 17 further comprising a needle cap that includes a flared portion of increasing diameter toward one end for engagement by the user during removal of the needle cap and a capping portion the frictionally fits upon said needle assembly.

20. The auto-injector of claim 17 further comprising button protecting means.
